(19) United States
(12) Patent Application Publication
(10) Pub. No.: US 2024/0390240 A1
(43) Pub. Date: Nov. 28, 2024

(54) COSMETIC PREPARATION CONTAINING LIME GLASS BEADS WITH HIGH SPECIFIC WEIGHT

(71) Applicant: BEIERSDORF AG, Hamburg (DE)

(72) Inventors: Berkay Daniel SIVACILAR, Hamburg (DE); Alexis GOULET-HANSSENS, Hamburg (DE); Heike VOLBRICH, Hamburg (DE); Carsten HEINSEN, Barmstedt (DE); David SCHLENKER, Hamburg (DE)

(21) Appl. No.: 18/693,229

(22) PCT Filed: Sep. 15, 2022
(86) PCT No.: PCT/EP2022/075632

§ 371 Date: Mar. 19, 2024

(30) Foreign Application Priority Data
Sep. 23, 2021 (DE) 10 2021 210 629.3

(51) Int. Cl. A61K 8/25; A61Q 19/00

(52) U.S. Cl. CPC A61K 8/25; A61Q 19/00; A61K 2800/30; A61K 2800/412; A61K 2800/651

(57) ABSTRACT

Cosmetic preparation containing at least one oil phase and soda-lime glass beads having a specific weight of at least 2.48 g/cm³.

The present invention relates to a cosmetic preparation comprising at least one oil phase and soda-lime glass beads having a density of at least 2.48 g/cm³.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skin cleansing is generally effected with the aid of surface-active preparations (soaps, surfactants, exceptionally alcoholic preparations) which are in the form of foam-forming gels or solids (soap bars) and after application to the skin are rinsed off again with water.

Skin care products, generally creams, ointments or lotions, mostly serve for moisturizing and refatting the skin. Active ingredients are commonly added thereto, which are intended to regenerate the skin and for example to prevent and reduce the premature aging thereof (e.g. the appearance of fine lines and wrinkles).

The plethora of commercially available skin care products, which also include sunscreens, should not however obscure the fact that these preparations from the prior art have a series of disadvantages.

To be accepted by consumers, cosmetic skin care products have to possess certain sensory properties. Tactile properties in particular play a central role in skin treatment. Thus, the preparations should ideally not draw any strings when applied and spread onto skin, but should be able to be easily rubbed and spread on the skin. Furthermore, the preparations should not leave any white residues on the skin after being rubbed in, i.e. they should “whiten” as little as possible.

It was therefore the object of the present invention to develop a cosmetic skin care product in which the “stringiness” and “whitening” is significantly reduced compared to preparations of the prior art.

One possible solution according to the prior art for reducing “stringiness” is to incorporate so-called powder raw materials, composed of plastic particles solid at room temperature, into the preparations. These plastic particles solid at room temperature consist of polyethylene, polypropylene, polymethyl methacrylate or nylon. This is also the definition according to the invention for these substances. The disadvantage now of this prior art is that the use of these substances is increasingly considered as undesirable in the context of the so-called “microplastic” discussion, i.e., the discussion around the use of plastic particles in cosmetics and the persistence thereof in the environment after application of cosmetics. Whether and potentially to what extent this discussion is justified need not be addressed in the context of this disclosure. The fact is, however, that there is an increasing interest among consumers in cosmetic products which no longer comprise these ingredients.

It was therefore the object of the present invention to improve the tactile properties of cosmetic skin care compositions without using plastic particles solid at room temperature, i.e., polyethylene, polypropylene, polymethyl methacrylate or nylon.

Surprisingly, these objects are achieved by a cosmetic preparation containing at least one oil phase and glass beads of soda-lime glass beads having a specific weight (density) of at least 2.48 g/cm³.

In this case, it is preferable in accordance with the invention if the preparation is free of plastic particles solid at room temperature.

The solution was particularly surprising, since the glass beads as a raw material are a white powder and a person skilled in the art had to assume that this property causes the preparation to “whiten” when applied to the skin, since the glass beads do not dissolve in the preparation.

It is the case that the prior art acknowledges cosmetic preparations having soda-lime glass beads in cosmetic preparations. For instance, DE10 2017 201869.0 discloses preparations with glass beads of soda-lime borosilicate. However, these have a significantly lower density (0.1-1.0 g/cm³) than the glass beads according to the invention. As tests with sensory-trained subjects have shown, these preparations tend to form more strings and to whiten more than the glass beads according to the invention.

It is advantageous in accordance with the invention if the glass beads according to the invention have a particle diameter of 3 to 110 μm. Preferred according to the invention are glass beads having a particle diameter of 3 to 20 μm or of 70 to 110 μm.

The particle size (particle diameter) of the glass beads according to the invention can be determined in this case in accordance with customary standards, for example by sieving. Of course, it is known to those skilled in the art that the particle size (grain size) is always subject to a certain size distribution. The size specification therefore signifies that no larger glass beads are used than those having a particle diameter of 110 μm and it is not critical if minor amounts of smaller particles are present in the preparation as impurities (for example formed by “broken glass”).

It is advantageous in accordance with the invention if the glass beads have a melting point of above 700° C. In this case, a melting point of about 730° C. is preferred in accordance with the invention. The borosilicate glass beads from 3M (Glass Bubbles K15) disclosed in DE10 2017 201869.0, in contrast, have a significantly lower melting point/softening temperature of 550 to 650° C.

Moreover, it is advantageous in accordance with the invention if the glass beads have an oil absorption of less than 0.2 grams of oil per cubic centimeter of glass beads, measured in accordance with ASTM D281-95.

Furthermore, the embodiments of the present invention that are advantageous according to the invention are characterized in that the glass beads have a refractive index of 1.4 to 1.6. In this case, a refractive index of 1.50-1.52 is preferred in accordance with the invention and a refractive index of 1.51 is particularly preferred.

As glass beads advantageous according to the invention, the glass beads “GP Cosmetics” from Kuhmichel or the glass beads “P2011SL Glass Microspheres” from Prizmalite may be used.

It is of advantage in accordance with the invention if the preparation comprises the glass beads in an amount of 0.1 to 5% by weight, based on the total weight of the preparation. In this case, a content of 1 to 2.5% by weight, based on the total weight of the preparation, is preferred in accordance with the invention.

Embodiments of the present invention that are advantageous according to the invention are characterized in that the oil phase of the preparation comprises one or more oil components selected from the group of the compounds diisopropyl adipate, caprylic/capric triglyceride (INCI Caprylic/Capric Triglycerides), isopropyl palmitate, dimethicone, cyclomethicone, octyldodecanol, ethylhexyl cocoate, myristyl myristate, hydrogenated coco-glycerides (INCI Hydrogenated Coco-Glycerides), dicaprylyl carbonate, C18-38 alkyl hydroxystearoryl stearate (INCI C18-38 Alkyl Hydroxystearoyl Stearate), di-n-butyl adipate, butylene glycol dicaprylate/dicaprate (INCI Butylene Glycol Dicaprylate/Dicaprate), C12-15 alkyl benzoate (INCI C12-15 Alkyl Benzoate), 2-phenylethyl benzoate (INCI Phenethyl Benzoate), di-C12-13 alkyl tartrate (INCI Di-C12-13 Alkyl Tartrate), butylene glycol cocoate (INCI: Butylene Glycol Cocoate), dicaprylyl ether, isodecyl neopentanoate, tridecyl trimellitate, isopropyl stearate, C12-13 alkyl lactate, 2-propylheptyl octanoate, isopropyl lauryl sarcosinate.

The oil components preferred in accordance with the invention are in this case diisopropyl adipate, caprylic/capric triglyceride (INCI Caprylic/Capric Triglycerides), octyldodecanol, ethylhexyl cocoate, myristyl myristate, hydrogenated coco-glycerides (INCI Hydrogenated Coco-Glycerides), di-n-butyl adipate, butylene glycol dicaprylate/dicaprate (INCI Butylene Glycol Dicaprylate/Dicaprate), C12-15 alkyl benzoate (INCI C12-15 Alkyl Benzoate), 2-phenylethyl benzoate (INCI Phenethyl Benzoate).

It is advantageous in accordance with the invention if the total amount of these oil components that are advantageous in accordance with the invention is from 1 to 20% by weight, based on the total weight of the preparation, the preferred amount range of which is from 2 to 15% by weight, based on the total weight of the preparation.

It is advantageous in accordance with the invention if the preparation according to the invention comprises one or more UV filters selected from the group of the compounds phenylene-1,4-bis(2-benzimidazyl)-3,3′-5,5′-tetrasulfonic acid salts; 2-phenylbenzimidazole-5-sulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl) sulfonic acid salts; 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene) camphor; 3-benzylidenecamphor; ethylhexyl salicylate; terephthalidenedicamphorsulfonic acid; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate; 4-(tert-butyl)-4′-methoxydibenzoylmethane; homomenthyl salicylate; 2-ethylhexyl 2-hydroxybenzoate; 2-ethylhexyl 2-cyano-3,3-diphenylacrylate; dimethicodiethylbenzalmalonate; 2-ethylhexyl 2-cyano-3,3-diphenylacrylate; 3-(4-(2,2-bis(ethoxycarbonylvinyl) phenoxy) propenyl) methoxysiloxane/dimethylsiloxane-copolymer; dioctylbutylamidotriazone (INCI: Diethylhexyl Butamidotriazone); 2,4-bis [5-1 (dimethylpropyl)benzoxazol-2-yl-(4-phenyl)imino]-6-(2-ethylhexyl)imino-1,3,5-triazine with (CAS No. 288254-16-0); tris(2-ethylhexyl) 4,4′,4″-(1,3,5-triazine-2,4,6-triyltriimino)trisbenzoate (also: 2,4,6-tris [anilino-(p-carbo-2′-ethyl-1′-hexyloxy)]-1,3,5-triazine (INCI: Ethylhexyl Triazone); 2,4-bis {[4-(2-ethylhexyloxy)-2-hydroxy]phenyl}-6-(4-methoxyphenyl)-1,3,5-triazine; 4-dicyanomethylene-2,6-dimethyl-1,4-dihydropyridine N-(ethyloxysulfate ester salts), titanium dioxide; zinc oxide.

It is preferred in accordance with the invention if the preparation according to the invention is free of 3-(4-methylbenzylidene) camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

In contrast, particular preference in accordance with the invention is given to the use of 4-(tert-butyl)-4′-methoxydibenzoylmethane, hexyl 2-(4′-diethylamino-2′-hydroxybenzoyl)benzoate (INCI: Diethylamino Hydroxybenzoyl Hexyl Benzoate), 2-phenylbenzimidazole-5-sulfonic acid salts, ethylhexyl salicylate; dioctylbutylamidotriazone (INCI: Diethylhexyl Butamidotriazone), tris(2-ethylhexyl) 4,4′,4″-(1,3,5-triazine-2,4,6-triyltriimino)trisbenzoate (also: 2,4,6-tris [anilino-(p-carbo-2′-ethyl-1′-hexyloxy)]-1,3,5-triazine (INCI: Ethylhexyl Triazone) and 2,4-bis {[4-(2-ethylhexyloxy)-2-hydroxy]phenyl}-6-(4-methoxyphenyl)-1,3,5-triazine, wherein these UV filters can be used singly or as a mixture.

Embodiments of the present invention that are advantageous according to the invention are characterized in that the preparation is in the form of an emulsion. In this case, O/W emulsions (oil-in-water emulsions) are preferred in accordance with the invention.

In such a case, it is particularly advantageous in accordance with the invention if the preparation is in the form of an O/W emulsion, which has been emulsified with one or more emulsifiers selected from the group of the compounds glyceryl stearate citrate, glyceryl stearate (self-emulsifying), stearic acid, stearate salts, polyglyceryl-3 methylglycose distearate, sodium stearoyl glutamate, sodium cetearyl sulfate, potassium cetyl phosphate, cetearyl sulfosuccinate, polyglyceryl-10 stearate, polyglyceryl-10 laurate, polyglyceryl-10 caprate. These emulsifiers are typically used at a total concentration of 0.5 to 10% by weight, based on the total weight of the preparation.

In the context of the present invention, it is advantageous if the preparation comprises one or more alcohols selected from the group of the compounds ethanol, glycerin ethylhexylglycerin, phenoxyethanol, polyglyceryl-2 caprate, propylene glycol, butylene glycol, 2-methylpropane-1,3-diol, 1,2-pentanediol, 1,2-hexanediol, 1,2-octanediol, 1,2-decanediol.

These alcohols may be used at a total concentration of 0.2 to 10% by weight, based on the total weight of the preparation.

Embodiments of the present invention that are advantageous according to the invention are characterized in that the preparation comprises one or more compounds selected from the group of the compounds alpha-lipoic acid, folic acid, phytoene, D-biotin, coenzyme Q10, alpha-glucosylrutin, carnitine, carnosine, isoflavonoids, flavonoids, creatine, creatinine, taurine, β-alanine, panthenol, polidocanol, tocopheryl acetate, dihydroxyacetone, glycerylglucose, (2-hydroxyethyl) urea, vitamin E and vitamin E acetate, hyaluronic acid and/or sodium salts thereof, menthol, glycyrrhetic acid and/or licochalcone A.

In addition, it is advantageous according to the invention if the preparation comprises ethanol and/or ethylhexylglycerin. In particular, the use of ethylhexylglycerin is preferred according to the invention.

Last but not least, it is advantageous in accordance with the invention if the preparation comprises one or more polymers selected from the group of the compounds xanthan gum, tapioca starch, hydroxyethylcellulose, vinylpyrrolidone/hexadecene copolymer.

In addition, the cosmetic preparation can be composed in the manner of a cosmetic customary for such cases and may comprise the appropriate known ingredients.

### Comparative Experiment

The following formulations were prepared and the “stringiness” and “whitening” after application to the skin were determined by a panel of 33 female test subjects trained for this purpose. The use amounts of the ingredients are stated in % by weight.

### Formulations

| | | Standard | Standard |
|---|---|---|---|
| | | with 2.5% | with 2.5% by |
| | Standard | by weight | weight GP |
| | without | P2011SL | Cosmetics |
| | glass | Prizmalite | 70-100 μm |
| INCI | beads | Glass Beads | Glass Beads |
| (P2011SL SODA LIME | | 2.5 | |
| GLASS MICROSPHERES) | | | |
| (GLASS BEADS-GP | | | 2.5 |
| COSMETICS 70-100) | | | |
| Ethylhexylglycerin | 0.25 | 0.25 | 0.25 |
| Dibutyl Adipate | 3 | 3 | 3 |
| C12-15 Alkyl | 5 | 5 | 5 |
| Benzoate | | | |
| Butylene Glycol | 4 | 4 | 4 |
| Dicaprylate/Dicaprate | | | |
| Glyceryl Stearate | 1 | 1 | 1 |
| Sodium Stearoyl | 0.3 | 0.3 | 0.3 |
| Glutamate + | | | |
| Sodium Chloride | | | |
| Perfume | 0.5 | 0.5 | 0.5 |
| Glycerin | 8.6 | 8.6 | 8.6 |
| Aqua + Sodium | 0.33 | 0.33 | 0.33 |
| Hydroxide | | | |
| Phenoxyethanol + | 0.5 | 0.5 | 0.5 |
| Aqua | | | |
| Aqua + Trisodium | 1 | 1 | 1 |
| EDTA | | | |
| Alcohol Denat. + | 6 | 6 | 6 |
| Aqua | | | |
| Stearyl Alcohol | 1 | 1 | 1 |
| Acrylates/C10-30 | 0.1 | 0.1 | 0.1 |
| Alkyl Acrylate | | | |
| Crosspolymer | | | |
| Xanthan Gum | 0.4 | 0.4 | 0.4 |
| Aqua | 56.27 | 53.77 | 53.77 |
| Ethylhexyl | 4 | 4 | 4 |
| Salicylate | | | |
| Bis-Ethylhexyloxyphenol | 1.75 | 1.75 | 1.75 |
| Methoxyphenyl Triazine | | | |
| Ethylhexyl Triazone | 1.5 | 1.5 | 1.5 |
| Butyl | 3.5 | 3.5 | 3.5 |
| Methoxydibenzoylmethane | | | |
| Phenylbenzimidazole | 1 | 1 | 1 |
| Sulfonic Acid | | | |

### Stringiness (Cohesiveness)

Stringiness is determined by assessing how long the string that a preparation applied to the palm forms between the finger and the palm when they are pulled apart before it breaks. The criteria range from “no string formation” to “forms a very long string” and are perceived visually. A total of 33 trained female test subjects were presented with a randomized and balanced order of products.

The descriptive statistics are presented as a tabular representation of the median for the degree of difference.

The inductive statistics enabled product comparisons by means of paired t-tests (two-sided hypothesis test, significance=0.1 and 0.05).

### Results

| | Standard + 2.5% by | Standard + 2.5% by |
|---|---|---|
| | weight P2011SL | weight GP Cosmetics |
| Comparison to the standard | Prizmalite Glass Beads | 70-100 μm Glass Beads |
| Stringiness | Significant improvement | Significant improvement |

*Significant change (p-value ≤ 5%) for the product with the higher intensity.

### EXAMPLES

The examples which follow are intended to illustrate the present invention without limiting it. Unless otherwise indicated, all amounts, proportions and percentages are based on the weight and the total amount or on the total weight of the preparations.

| | Examples | | | |
|---|---|---|---|---|
| Materials | Example 1 | Example 2 | Example 3 | Example 4 |
| P2011SL | 2.5 | — | 2.5 | — |
| Prizmalite Glass | | | | |
| Beads (%) | | | | |
| GP Cosmetics | — | 2.5 | — | 2.5 |
| 70-100 μm Glass | | | | |
| Beads (%) | | | | |
| Water (%) | 97.15 | 97.15 | 96.1 | 85.3 |
| Phenoxyethanol | 0.2 | 0.2 | 0.2 | 0.2 |
| (%) | | | | |
| Gellan Gum (%) | 0.15 | 0.15 | — | — |
| Xanthan Gum (%) | — | — | 1.2 | 2.0 |
| Glycerin (%) | — | — | — | 10.0 |

1.-16. (canceled)

17. A cosmetic preparation, wherein the preparation comprises at least one oil phase and soda-lime glass beads having a density of at least 2.48 g/cm³.

18. The cosmetic preparation of claim 17, wherein the preparation is free of plastic particles solid at room temperature.

19. The cosmetic preparation of claim 17, wherein the glass beads have a particle diameter of from 3 μm to 110 μm.

20. The cosmetic preparation of claim 17, wherein the glass beads have a particle diameter of from 3 μm to 20 μm.

21. The cosmetic preparation of claim 17, wherein the glass beads have a particle diameter of from 70 μm to 110 μm.

22. The cosmetic preparation of claim 17, wherein the glass beads have a melting point of higher than 700° C.

23. The cosmetic preparation of claim 17, wherein the glass beads have an oil absorption of less than 0.2 grams of oil per cubic centimeter of glass beads, measured in accordance with ASTM D281-95.

24. The cosmetic preparation of claim 17, wherein the glass beads have a refractive index of from 1.4 to 1.6.

25. The cosmetic preparation of claim 17, wherein the glass beads have a refractive index of from 1.50 to 1.52.

26. The cosmetic preparation of claim 17, wherein the preparation comprises the glass beads in a concentration of from 0.1% to 5% by weight, based on a total weight of the preparation.

27. The cosmetic preparation of claim 17, wherein the preparation comprises the glass beads in a concentration of from 1% to 2.5% by weight, based on a total weight of the preparation.

28. The cosmetic preparation of claim 17, wherein the at least one oil phase of the preparation comprises one or more components selected from diisopropyl adipate, caprylic/capric triglyceride (INCI Caprylic/Capric Triglycerides), isopropyl palmitate, dimethicone, cyclomethicone, octyldodecanol, ethylhexyl cocoate, myristyl myristate, hydrogenated coco-glycerides (INCI Hydrogenated Coco-Glycerides), dicaprylyl carbonate, C18-38 alkyl hydroxystearoryl stearate (INCI C18-38 Alkyl Hydroxystearoyl Stearate), di-n-butyl adipate, butylene glycol dicaprylate/dicaprate (INCI Butylene Glycol Dicaprylate/Dicaprate), C12-15 alkyl benzoate (INCI C12-15 Alkyl Benzoate), 2-phenylethyl benzoate (INCI Phenethyl Benzoate), di-C12-13 alkyl tartrate (INCI Di-C12-13 Alkyl Tartrate), butylene glycol cocoate (INCI: Butylene Glycol Cocoate), dicaprylyl ether, isodecyl neopentanoate, tridecyl trimellitate, isopropyl stearate, C12-13 alkyl lactate, 2-propylheptyl octanoate, isopropyl lauryl sarcosinate.

29. The cosmetic preparation of claim 17, wherein the preparation further comprises one or more UV filters selected from phenylene-1,4-bis-(2-benzimidazyl)-3,3′-5,5′-tetrasulfonic acid salts; 2-phenylbenzimidazole-5-sulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl) sulfonic acid salts; 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene) camphor; 3-benzylidenecamphor; ethylhexyl salicylate; terephthalidenedicamphorsulfonic acid; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate; 4-(tert-butyl)-4′-methoxydibenzoylmethane; homomenthyl salicylate; 2-ethylhexyl 2-hydroxybenzoate; 2-ethylhexyl 2-cyano-3,3-diphenylacrylate; dimethicodiethylbenzalmalonate; 2-ethylhexyl 2-cyano-3,3-diphenylacrylate; 3-(4-(2,2-bis(ethoxycarbonylvinyl) phenoxy) propenyl)-methoxysiloxane/dimethylsiloxane-copolymer; dioctylbutylamidotriazone (INCI: Diethylhexyl Butamidotriazone); 2,4-bis [5-1 (dimethylpropyl)benzoxazol-2-yl-(4-phenyl)imino]-6-(2-ethylhexyl)imino-1,3,5-triazine with (CAS No. 288254-16-0); tris(2-ethylhexyl) 4,4′,4″-(1,3,5-triazine-2,4,6-triyltriimino)trisbenzoate (also: 2,4,6-tris [anilino-(p-carbo-2′-ethyl-1′-hexyloxy)]-1,3,5-triazine (INCI: Ethylhexyl Triazone); 2,4-bis {[4-(2-ethylhexyloxy)-2-hydroxy]phenyl}-6-(4-methoxyphenyl)-1,3,5-triazine; 4-dicyanomethylene-2,6-dimethyl-1,4-dihydropyridine N-(ethyloxysulfate ester salts), titanium dioxide; zinc oxide.

30. The cosmetic preparation of claim 17, wherein the preparation is present in the form of an emulsion.

31. The cosmetic preparation of claim 17, wherein the preparation is present in the form of an O/W emulsion, which has been emulsified with one or more emulsifiers selected from glyceryl stearate citrate, glyceryl stearate (self-emulsifying), stearic acid, stearate salts, polyglyceryl-3 methylglycose distearate, sodium stearoyl glutamate, sodium cetearyl sulfate, potassium cetyl phosphate, cetearyl sulfosuccinate, polyglyceryl-10 stearate, polyglyceryl-10 laurate, polyglyceryl-10 caprate.

32. The cosmetic preparation of claim 17, wherein the preparation further comprises one or more alcohols selected from ethanol, glycerin, ethylhexylglycerin, phenoxyethanol, polyglyceryl-2 caprate, propylene glycol, butylene glycol, 2-methylpropane-1,3-diol, 1,2-pentanediol, 1,2-hexanediol, 1,2-octanediol, 1,2-decanediol.

33. The cosmetic preparation of claim 17, wherein the preparation further comprises one or more compounds selected from alpha-lipoic acid, folic acid, phytoene, D-biotin, coenzyme Q10, alpha-glucosylrutin, carnitine, carnosine, isoflavonoids, flavonoids, creatine, creatinine, taurine, β-alanine, panthenol, polidocanol, tocopheryl acetate, dihydroxyacetone, glycerylglucose, (2-hydroxyethyl) urea, vitamin E and vitamin E acetate, hyaluronic acid and/or sodium salts thereof, menthol, glycyrrhetic acid and/or licochalcone A.

34. The cosmetic preparation of claim 17, wherein the preparation further comprises one or more polymers selected from xanthan gum, tapioca starch, hydroxyethylcellulose, vinylpyrrolidone/hexadecene copolymer.

35. The cosmetic preparation of claim 17, wherein the preparation is free from 3-(4-methylbenzylidene) camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers, polyethylene glycol esters.

36. The cosmetic preparation of claim 17, wherein the preparation further comprises ethanol and/or ethylhexylglycerin.
